(19) United States
(12) Patent Application Publication
(10) Pub. No.: US 2012/0294898 A1
(43) Pub. Date: Nov. 22, 2012

(54) INJECTABLE DBM FOR SOFT TISSUE REPAIR

(75) Inventors: William G. Hubbard, Burlington, WI (US); Christina Mossaad, Holly Springs, NC (US); Lawrence A. Shimp, Morganville, NJ (US); Hsiu Ying Sherry Wang, North Brunswick, NJ (US)

(73) Assignee: Warsaw Orthopedic, Inc., Warsaw, IN (US)

(21) Appl. No.: 13/111,541

(22) Filed: May 19, 2011

(51) Int. Cl. A61K 9/14; A61P 19/00; A61K 38/18

(52) U.S. Cl. 424/400; 514/7.6

(57) ABSTRACT

An injectable pharmaceutical composition includes demineralized bone matrix (DBM) particles having a particle size in the range of about 25 microns to about 75 microns and a pharmaceutical carrier. The composition can further include adjuncts such as glycosaminoglycans, adjuvants such as chondroitin, and/or active ingredients such as anti-inflammatories.

### FIELD OF THE DISCLOSURE

This disclosure, in general, relates to a medical composition for cartilage regeneration and/or joint disease and methods of making and using the medical composition.

### BACKGROUND

Joint disease involves damage to the joint cartilage and results in joint pain and other symptoms related to this disease state. In mild cases, the symptoms can be treated to provide some relief, without addressing the underlying cause. A common treatment for symptoms of joint pain often includes injection of a lubricant into the joint. Hyaluronic acid is a commonly used lubricant, as it is compositionally similar to original synovial fluid. Such a treatment can relieve pain by flushing out inflammatory molecules (e.g., cytokines) as well as by its actual lubrication properties. A permanent cure requires either regeneration of new cartilage, which is not possible in most cases, or replacement with an artificial joint. Cartilage regeneration can sometimes be brought about by nutritional supplements containing glucosamine and chondroitin sulfate, which are building blocks of natural cartilage. However, the benefits are limited, and not all patients see an improvement.

Alternate surgical approaches include micro-fracture surgery where the damaged area of cartilage is removed, and bleeding bone is exposed. The theory is that cells will migrate from the bleeding bone and form new cartilage. This surgical procedure can be supplemented by placement of a scaffold of collagen, demineralized bone matrix (DBM) flexible sheet, or a polymer scaffold on the site to induce cell growth. Another approach is to transplant plugs of healthy cartilage from the edge of the joint surface to the injured site. Other approaches include use of cultured allograft cells or growing cartilage cells from the patient and placing them in the defect or injured site.

Avoiding surgery, if possible, is an advantage; but any nonsurgical technique should be more effective, in terms of cartilage regeneration, than just adding a simple lubricant. As an injected lubricant has contact with the injured cartilage, it could be used to deliver nutrients and growth factors. One such non-surgical approach involves injecting a growth factor, mixed into the lubricant, into the damaged joint to take advantage of chondrocyte-stimulating properties of the growth factor in an attempt to regenerate new tissue. Growth factors used in such a treatment include insulin or its derivatives, or sulfated polysaccharides. These materials are not directly active, but hold and concentrate naturally occurring growth factors. It is also possible to inject anti-inflammatory compounds to reduce the extent of injury, or other materials that inhibit cartilage damage.

Such techniques can be effective, but are not considered to be long-term solutions to cartilage damage associated with joint disease. As a consequence, there remains an on-going search to provide a more immediate approach to cartilage regeneration without resorting to invasive surgical procedures. As such, an improved medical composition is desired.

### SUMMARY

In an embodiment, an injectable pharmaceutical composition includes demineralized bone matrix (DBM) particles having a particle size in the range of about 25 microns to about 75 microns and a pharmaceutical carrier.

In another exemplary embodiment, a method for treating connective tissue disease or regenerating cartilage includes identifying indications and location of a connective tissue disease or cartilage diminution in a patient, providing a pharmaceutical composition including demineralized bone matrix (DBM) particles having a particle size in the range of about 25 microns to about 75 microns, and injecting the pharmaceutical composition at or near the location of connective tissue disease or cartilage diminution.

In a particular embodiment, a pharmaceutical composition includes demineralized bone matrix (DBM) particles at a particle size between about 25 to about 50 microns, type 2 collagen, and hyaluronic acid. The pharmaceutical composition can be formulated in an aqueous medium.

In another embodiment, a controlled release formulation for releasing growth factors includes DBM particles having a particle size in the range of about 25 microns to about 75 microns, wherein the DBM has at least one natural growth factor and a controlled release rate of the at least one natural growth factor.

### DETAILED DESCRIPTION

It is an object of the present invention to provide a non-surgical, early stage approach to cartilage regeneration, together with related compositions and methods, thereby overcoming various deficiencies and shortcomings of the prior art, including those outlined above. It would be understood by those skilled in the art that one or more aspects of this invention can meet certain objectives, while one or more aspects can meet certain other objectives. Each objective may not apply equally, in all its respects, to every aspect of this invention. As such, the following objects can be viewed in the alternative with respect to any one aspect of this invention. It can be an object of the present invention to provide a methodology for treatment of joint disease and related cartilage damage to better utilize the benefits and advantages associated with DBM, without resort to invasive surgery.

It can also be an object of the present invention to provide a near-term approach to cartilage regeneration, in conjunction with treatment of symptomatic joint damage, inflammation or infections at or near the joint, or pain associated therewith. It can also be an object of the present invention to provide a near-term approach to tendon or ligament regeneration, in conjunction with treatment of inflammation, infections, or pain from injured tendons or ligaments.

It can also be an object of the present invention, alone or in conjunction with one or more of the preceding objectives, to provide a therapeutic composition including DBM and one or more adjuncts, pharmaceutical carrier, optionally including one or more adjuvants and/or pharmaceutical agents to further promote cartilage and/or tendon and ligament regeneration.

Other objects, features, benefits and advantages of the present invention will be apparent from the following descriptions of certain embodiments, and will be readily apparent to those skilled in the area of joint disease, cartilage regeneration, and connective tissue injuries. Such objects, features, benefits and advantages will be apparent from the above as taken into conjunction with the accompanying examples, data, and all reasonable inferences to be drawn therefrom, alone or with consideration of the references incorporated herein.

The present invention provides an advantage over existing treatments in that it provides compositions of DBM and methods of treatment that are suitable for application to soft tissue. As used herein, soft tissue includes but is not limited to ligaments, tendons, hernias, spinal disks, and joints. This provides a non-surgical, early stage approach to cartilage regeneration. The technology can also be used to repair ligaments and tendons by an injection containing DBM material.

In a particular embodiment, an injectable pharmaceutical composition includes demineralized bone matrix (DBM) particles. The DBM particles naturally provide a scaffold and growth factors for cartilage regeneration and joint repair. In an exemplary embodiment, the DBM particles have a particle size in the range of about 25 microns to about 75 microns. In particular, the particle size of the DBM provides a composition that can be percutaneously injected into a patient. Accordingly, this DBM composition provides a non-surgical approach for cartilage regeneration. Furthermore, the injectable pharmaceutical composition has a pharmaceutical carrier. In another embodiment, the DBM particles are present in the form of a suspension in the injectable pharmaceutical composition. The suspension can be formed by any conventional method available.

In an embodiment, the DBM particles can have a particle size in the range of about 25 microns to about 50 microns. In another embodiment, the range can be from about 25 microns to about 35 microns. In another embodiment, the size of DBM particles is not greater than about 50 microns, not greater than about 40 microns, or not greater than about 30 microns. Furthermore, the composition can have DBM particles of a size of less than 25 microns. For example, the DBM particles can have a particle size of at least about 0.5 micron, at least about 1 micron, at least about 2 microns, at least about 5 microns, at least about 10 microns, at least about 15 microns, or at least about 20 microns.

The DBM particle size is estimated from the measurement of the length (maximum dimension) and width (minimum dimension) of a large number of single particles (at least 20 particles randomly chosen). The particle size can be averaged using the widths, lengths, or a combination thereof, for example an average of the average width and average length (i.e., (average width+average length)/2).

In one embodiment, the DBM particles are present from about 1 wt % to about 99 wt % of the pharmaceutical composition. In another embodiment, the DBM particles are present from about 1 wt % to about 50 wt % of the pharmaceutical composition. In another embodiment, the DBM particles are present from about 1 wt % to about 25 wt % of the pharmaceutical composition. In another embodiment, the DBM particles are present from about 1 wt % to about 10 wt % of the pharmaceutical composition. In still other embodiments, the DBM particles are present from about 1 wt % to about 5 wt %, about 1 wt % to about 4 wt %, about 1 wt % to about 3 wt %, about 1 wt % to about 2 wt % of the pharmaceutical composition. In another embodiment, the DBM particles are present at least about 1 wt %, at least about 2 wt %, at least about 3 wt %, at least about 5 wt %, at least about 10 wt %, or at least about 20 wt % of the pharmaceutical composition. In another embodiment the DBM particles form not greater than about 99 wt %, not greater than about 75 wt %, not greater than about 50 wt %, not greater than about 25 wt %, not greater than about 15 wt %, or not greater than about 10 wt % of the pharmaceutical composition.

The DBM can be selected from intact DBM, digested DBM, partially digested DBM, and combinations thereof. Intact DBM is a demineralized bone matrix obtained commercially or can be prepared by techniques known in the art. In general, DBM material can be prepared by demineralization or decalcification of bone by acid treatment. The technique can be modified to leave collagen, proteins, and natural growth factors in a solid matrix. The DBM can be derived from any animal source, including human. In an embodiment, human DBM can also be genetically engineered from animal sources. For example, pig DNA encoding this animal's bone can be modified to yield human bone material from which human DBM is obtained. The modification of the animal DNA includes replacing at least one nucleotide of the bone or DBM gene from the animal. Alternatively, sequences of nucleotides bone or DBM genes from pig can be replaced to form human bone or DBM material. Alternatively, animal bone, for example, pig bone can serve as base material for human-compatible DBM. Such DBM has an animal origin but functions equally to human DBM and does not cause any or only a minimal immune response in the patient. In a particular embodiment, genetically modified animal DNA provides a source of DBM that does not have an immunological response in comparison to non-human bone material that has not been genetically modified.

In embodiment, it can be advantageous to first grind or otherwise reduce the DBM particle size to under 1 mm, preferably under 100 microns before exposing the DBM to further treatment. In another embodiment, bone material can first be reduced in size by breaking, crushing, grinding, or milling bones, followed by demineralization of the resulting material.

To increase its effectiveness, DBM can be digested, or partially digested with any reasonable enzyme that at least partially preserves the activity of associated natural growth factors contained within the DBM. In a particular embodiment, the DBM can be digested, or partially digested with a collagenase enzyme such that the activity of associated natural growth factors is at least partially preserved. Advantageously, the digested material is more readily available to the injured tissue and therefore more effective in promoting repair or regeneration processes of cartilage and connective tissue. Alternatively, digested DBM or partially digested DBM can be combined with intact DBM powder. Combining intact DBM with digested variations provide both short-term and long-term activity.

The amount of digestion of the DBM affects the rate of release of proteins embedded in the DBM into the surrounding environment. Such proteins are, for example, growth factors, which are essential in tissue generation and tissue growth. Releasing natural growth factors from its natural tissue environment, such as from the DBM, ensures undiminished protein activity in comparison to isolated growth factors stored in a manufactured medium. Digested DBM provides for growth factors that are immediately released, while partially digested or undigested DBM provides for delayed release of growth factors. Accordingly, the degree of digestion and the mixing of fully or partially digested DBM with undigested DBM provides for a method to control the release of growth factors from DBM into the location of treatment.

In an embodiment, an injectable pharmaceutical formulation may include fully digested DBM combined with partially digested and/or undigested DBM. Upon administration into the site of cartilage regeneration, fully digested DBM releases growth factors that commences and assists with the cartilage regeneration process. Released growth factors from fully digested DBM are limited in their time of activity and subject to removal from the site of injury by natural wound healing or medical processes. However, the continuous stream of growth factors from partially digested or undigested DBM counters this effect and ensures continuous activity of growth factors at the site of injury.

In another embodiment, the injectable DBM formulation can have a growth factor release rate greater than the removal rate. Such formulation ensures a controlled overall increase of growth factors at the site of injury. This may be desired at stages where more growth factors are required as newly regenerated cartilage tissue increases in volume.

In an embodiment, the injectable pharmaceutical formulation includes a first DBM material containing at least one natural growth factor, the DBM material having a first controlled release rate of the at least one natural growth factor. In a particular embodiment, the amount of digestion of the DBM and growth factors contained within the DBM may be tailored depending on the desired release rate, desired result, and application. In an embodiment, the injectable pharmaceutical formulation further includes a second DBM material containing at least one natural growth factor, the second DBM material having a second controlled release rate of the at least one natural growth factor. In an embodiment, the first controlled release rate and the second controlled release rate are different. For instance, the first controlled release rate may be faster than the second controlled release rate. The at least one natural growth factor contained within the first DBM material may be the same or different than the at least one natural growth factor contained within the second DBM material. Any number of DBM materials with growth factors may be envisioned with any number of release rates for the growth factors. The obtained DBM material can be used as dry powder as a starting material for the pharmaceutical compositions. Alternatively, the obtained DBM can be milled into any desired particle size to prepare the pharmaceutical compositions. Alternatively, the DBM can be prepared as a suspension using an aqueous acid, such as lactic acid.

The pharmaceutical composition can include at least one natural growth factor selected from any member of the Transforming Growth Factor (TGF) superfamily, BMP-2, BMP-4 and BMP-7, transforming growth factor-β (TGF-β); platelet derived growth factor (PDGF); fibroblast growth factor (FGF); insulin-like growth factors (IGF); connectivity tissue-derived growth factors (CTGF); cartilage-derived growth factors (CDGF); and Vascular endothelial growth factor (VEGF). In embodiments, the pharmaceutical composition can include natural growth factors selected from BMP-2, IGF-1, VEGF, FGFa, TGF-β1, PDGF, and BMP-4.

The total amount of growth factor is present in a concentration of between about 10 picogram per gram DBM (“pg/g”) and about 10,000 nanogram per gram DBM (“ng/g”). In an embodiment, BMP-2 can be present between about 2000 ng/g and about 6000 ng/g. In another embodiment, IGF-1 can be present between about 10,000 pg/g and about 25,000 pg/g. In another embodiment, VEGF can be present between about 800 pg/g and about 1500 pg/g. In another embodiment, FGFa can be present between about 10,000 pg/g and about 30,000 pg/g. In yet another embodiment, TGF-β1 can be present between about 10 pg/g and about 10,000 pg/g. In another embodiment, PDGF can be present between about 50 pg/g and about 500 pg/g. In yet another embodiment, BMP-4 can be present between about 1 pg/g and about 100 pg/g.

As relates to various embodiments of this invention, the natural growth factors in demineralized bone are effective in growing cartilage. In particular, the collagen naturally present in DBM is also an effective scaffold for cartilage growth. Accordingly, this invention includes a nonsurgical technique to deliver DBM to a damaged joint by injection along with at least one adjunct, such as but not limited to a pharmaceutical carrier.

In embodiments, the injectable formulation may include adjuncts, adjuvants, and active ingredients. Adjuncts are compounds and composition that stabilize the DBM material. These adjuncts serve in a supportive role and may include matter that stabilizes growth factors in the DBM or collagen environment. Adjuvants are compounds or compositions that contribute to the regeneration process or replenish enzymes. For example, adjuvants may be additional growth factors or natural building blocks for cartilage such as chondroitin. By nature of some compositions, an adjunct may also serve as an adjuvant. An active ingredient is a pharmaceutically active compound that responds to a side reaction at the site of injury. For example, a site in need of cartilage regeneration may incur an inflammation or bacterial infection which is concurrently treated with an anti-inflammatory or antibiotic present in the DBM formulation.

In one embodiment, powdered DBM can be suspended in an adjunct, such as a lubricant fluid. The suspension can be injected into the joint or location where repair or regeneration of connective tissue or cartilage is required. Upon administration, DBM particles settle at the location where connective tissue or cartilage is required and begin to function as active scaffold. The adjunct can be a member of the glycosaminoglycan (GAG) group which includes hyaluronan, hyaluronic acid, or hyaluronate, or other suitable materials known to those skilled in the art made aware of this invention.

Notwithstanding DBM identity and/or dimension, the adjunct of the present compositions can be a glycosaminoglycan or combinations of such materials. In certain non-limiting embodiments, such an adjunct can be hyaluronic acid, a biomimetic substitute known in the art for synovial fluid, and combinations thereof. In an embodiment, the adjunct may be present in an amount of about 1% by weight to about 99% by weight of the total composition, such as about 1 wt % to about 5 wt %, or about 1 wt % to about 3 wt %. In other embodiments, synthetic or extracted synovial fluid can be used as an adjunct. If synovial fluid is used in the formulation, the amount therof would be much larger since synovial fluid has the desired consistency and natural ingredients in appropriate concentrations for treatment of injured cartilage or connective tissue. For example, the amount of synthetic or extracted synovial fluids is at least 10 wt %, such as at least 20 wt %, at least 40 wt %, at least 60 wt %, or even at least 80 wt % of the formulation.

For example, in other embodiments, the at least one adjunct can be selected from proteoglycans, one or more enzymes, proteins, deoxyribonucleic acids selected from genes, gene fragments and antisense DNA, ribonucleic acid selected from small interfering RNA (siRNA) or a microRNA, glucosamine and glucosamine derivatives, and chondroitin.

In an embodiment, the DBM may be suspended in a pharmaceutical carrier component. Any reasonable pharmaceutical carrier component may be envisioned. In a particular embodiment, the pharmaceutical carrier may be any reasonable biomimetic substitute for synovial fluid. In an embodiment, the pharmaceutical carrier is glycosaminoglycan or combinations of such materials. In certain other embodiments, a pharmaceutical carrier component can be selected from a saline solution in combination with an aqueous gelling agent. Any reasonable aqueous gelling agent may be envisioned. In a particular embodiment, the gelling agent can be selected from carbopol polymers, various alginates and combinations thereof. In an embodiment, the carrier component is present in an amount from about 1% by weight to about 99% by weight of the total composition, such as from about 50 wt % to about 90 wt %. Such compositions can include various other adjuvants and/or pharmaceutical agents. For instance, without limitation, such compositions can include chondroitin, glucosamine, one or more additional growth factors, various cellular matter, anti-inflammatory components and combinations thereof. Various other pharmaceutical agents and/or adjuvants will be known to those skilled in the art made aware of this invention. In other embodiments, aqueous hyaluronic solution at a concentration between 0.1 to 99 wt %, such as between about 0.1 and about 5 wt %, or between about 0.5 and about 2 wt % can serve as a carrier. The amount of the hyaluronic solution in the formulation can range between about 50% to about 90 wt % of the injectable formulation.

In another embodiment, the pharmaceutical composition is formulated to be a thixotropic composition, i.e., a formulation that has lowered viscosity when shaken or put under pressure and increases in viscosity upon administration and thereafter. Such compositions are beneficial as they permit precision during administration. For instance, the viscosity of the composition may be similar to the viscosity of synovial fluid in human joints. For example, the viscosity can be about 20 cP when shaken or put under pressure but increases to about 1000 cP after administered at the treatment location. Viscosity is measured by any conventional method. For instance, viscosity can be measured at room temperature (about 25° C.) with a Brookfield Viscometer or Rheometer such as the Brookfield DV-III Ultra CP Programmable Rheometer at various shear rates to determine the variation of viscosity of the thixotropic formulation.

In one aspect, the present invention can be directed to a cartilage regeneration composition including a DBM component and a glycosaminoglycan component. In a particular embodiment, the glycosaminoglycan component is hyaluronic acid. The DBM component can include particles dimensioned from about 25 microns to about 75 microns or as otherwise specified herein. In embodiments, a DBM component can be selected from digested DBM, partially digested DBM and intact DBM particles and combinations thereof. In some embodiments, the composition consists essentially of the respective DBM and glycosaminoglycan described above. As used herein, the phrase “consists essentially of” used in connection with the DBM and glycosaminoglycan precludes the presence of other components that affect the basic and novel characteristics of the DBM and glycosaminoglycan, although, commonly used carrier components may be used in the composition.

In addition to DBM, the composition can contain an adjuvant. An exemplary adjuvant is collagen obtained from any type of collagen, for example, animal, human, or genetically reproduced. In a particular embodiment, Type 1 collagen, Type 2 collagen, or a combination thereof have beneficial properties for the composition. In a particular embodiment, the collagen is Type 2 collagen. The collagen can be made by any method known to the art of collagen processing. In an embodiment, the collagen can be particles, fibers, or any combination thereof in a size advantageous for percutaneous injection. For instance, the collagen particles may be not greater than about 75 microns, such as between about 25 microns to about 50 microns. In a particular embodiment, the DBM and collagen are homogenously mixed in the pharmaceutical carrier. Typically, when an adjuvant is present, it is in an amount of about 1% by weight to about 10% by weight of the total composition, such as between about 1 to about 3 wt %, or between about 1 to about 2 wt %.

The pharmaceutical composition can have additionally at least one adjuvant such as the above-discussed collagen, cellular matter, chondroitin sulfate, glucoseamine and additional growth factor, polysaccharides, or growth factor peptides. Another useful class of optional additives is non-heparin sulfated polysaccharides, especially those derived from marine sources. Cellular matter can also be used in conjunction with the present compositions and methods. Such matter can include chondrocytes, human mesenchymal stem cells or other cells capable of differentiating to chrondrocytes. Additional growth factors can be selected from any member of the Transforming Growth Factor (TGF) superfamily, BMP-2, BMP-4 and BMP-7, transforming growth factor-β (TGF-β); platelet derived growth factor (PDGF); fibroblast growth factor (FGF); insulin-like growth factors (IGF); connectivity tissue-derived growth factors (CTGF); cartilage-derived growth factors (CDGF); and Vascular endothelial growth factor (VEGF). In embodiments, the pharmaceutical composition can include additional growth factors selected from BMP-2, IGF-1, VEGF, FGFa, TGF-β1, PDGF, and BMP-4.

Furthermore, the pharmaceutical composition can contain at least one active ingredient, wherein the ingredient can be an anti-inflammatory agent, an analgesic, an antibiotic, or an antioxidant. The anti-inflammatory agent can be any one of those listed above in connection with flushing the site of administration.

In embodiments, an anti-inflammatory agent can be selected from Omega-3 EFA, EPA, DHA, white willow bark, salicin, curcumin, epigallocatechin-3 galate, pycnogenol, olibanum, uncaria tomentosa, U. guianensis 100, capsaicin, ginger, glucocorticoids, corticosteroids, mannose-6-phosphate, heparin, castanospermine, licodelone, indomethacin, ibuprofin, aspirin, choline salicylate, difunisal, magnesium salicylate, magnesium choline salicylate, salsalate, flurbiprofen, fenoprofen, ketoprofen, naprosen, naproxen sodium, oxaprozin, diclofenac sodium, diclofenac misoprostol, etodolac, indocin, ketorolac, natumetone, sulindac, tolmetin, sulfinpyrazone, dipyridamole, ticlopidine, valdecoxib, rofecoxib, piroxicam, meloxicam, meclofenamate sodium, mefenamic, cyclophosphamide, cyclosporine micromulsion, chiorambucil, anagrelide, clopidogrel, galactose, vitamin D, cilostazol, and combinations thereof. Anti-inflammatory agents may be present in an amount sufficient to provide anti-inflammatory relief to the injection site.

Any reasonable analgesic can be included in the DBM composition. In an embodiment, the analgesic can be selected from morphine, heroin, hydromorphone, metopon, oxymorphone, levorphanol, codeine, hydrocodone, xycodone, nalorphine, naloxone, naltrexone and, salycilates, phenylbutazone, indomethacin, phenacetin, and combinations thereof.

Any reasonable antibiotics can be included in the DBM composition. In an embodiment, the antibiotic can be selected from one or more aminoglycosides, amphenicols, ansamycins, beta-lactams, lincosamides, macrolides, polypeptide antibiotics, tetracyclines, cycloserine, mupirocin, tuberin, 2,4-diaminopyrimidines, nitrofurans, quinolones, sulfonamides, sulfones, clofoctol, hexedine, methenamine, nitroxoline, taurolidine, xibernol, and combinations thereof.

In other embodiments, any reasonable antioxidant can be included in the DBM formulation. In an embodiment, the antioxidant can be selected from carotenoids, flavonoids, isoflavones, vitamins, coenzyme-Q10, glutathione, superoxide dismutase, and combinations thereof. In embodiments, the pharmaceutical composition can be administered through a needle having a gauge size between 14 and 23. The volume of administration depends from the site of treatment or joint. For example, an adult knee joint can be administered with a volume of the pharmaceutical composition between about 2.5 mL and 4 mL. In embodiments, the volume can be not greater than about 4.0 mL, not greater than about 3.5 mL, not greater than about 3.0 mL, not greater than about 2.5 mL, not greater than about 2.0 mL, not greater than about 1.5 mL, not greater than about 1.0 mL, or not greater than about 0.5 mL.

In another aspect, the present invention is a method for the non-surgical treatment of cartilage regeneration, using the DBM composition. Such a method can include providing a composition of the sort described above; and administering such a composition to a subject with indications of joint disease and attendant cartilage damage. Administration can include such a composition non-surgically positioned within a joint cavity, between articulating surfaces. Such a composition can include a carrier component concentration at least partially sufficient to affect joint lubrication and a DBM component concentration at least partially sufficient to provide a scaffold for or otherwise induced cartilage growth.

In an embodiment, administration of the composition includes any reasonable method envisioned. For instance, such a composition can be administered by injection needle for percutaneous injection. In certain such embodiments, a useful injection needle can have a nominal inner diameter of 1 mm or less or a gauge index of 23-gauge or more. In particular, the administration of the composition is typically through an injection needle sized such that the composition does not extravasate out of a patient's body from the injection site.

Before injecting the DBM composition to an injury site, it may be advantageous to flush the location of connective tissue or cartilage repair with saline to remove as many of the inflammatory molecules (e.g., cytokines) as possible. Reducing the inflammatory reaction will provide a more favorable environment for healing and to promote subsequent cartilage regeneration. As understood in the art, a saline flush not followed by application of a lubricant can lead to increased joint damage and pain. In addition to, or as a substitute to flushing, the composition can contain any reasonable anti-inflammatory agents as described above.

Applications of the present DBM compositions can be expanded to any injured soft tissue. Soft tissue includes, for example, tendons, ligaments, hernias, connective tissue, spinal disks, wound care, joints, and the like. In a particular embodiment, soft tissue is in contrast to hard tissue, i.e., cortical and cancellous bone. While a lubricant carrier is useful for joint applications, such as elbows, knees, hands, feet, and shoulders, it may not be necessary for all applications. For instance, saline or another carrier could be used. A gelling carrier is especially advantageous for joint and spinal disk applications, or any place where the possibility of migration needs to be minimized. Non-limiting examples of gelling carriers include carbopol polymers and certain alginates and chitosans. In an embodiment, the gelling carriers include, but are not limited to, carbopol polymers, polyethylene glycol, alginate, chitosan, pectin, chitin, or glycogen. In another embodiment, the formulation may be useful for reconstructing of cartilage and/or connective tissue in cosmetic surgery.

Commercially available DBM applications are typically used for hard tissue application such as bone void filler or bone gap filler, while formulations of the present composition are useful for the treatment of soft tissue.

### EXAMPLES

The following non-limiting examples and data illustrate various aspects and features relating to the compositions and methods of the present invention, for treatment of joint disease and cartilage regeneration with DBM, as are available through the DBM compositions described herein. In comparison with the prior art, the present methods and compositions provide results and data which are surprising, unexpected and contrary thereto. While the utility of this invention is illustrated through the use of several compositions and corresponding DBM and carrier components, it will be understood by those skilled in the art that comparable results are obtainable with various other compositions and components, in conjunction with associated treatment methodologies, as are commensurate with the scope of this invention.

### Example 1

In the various embodiments, powdered DBM is suspended in concentrations of about 1 wt. % to about 99 wt. % in a lubricant fluid, and injected with the lubricant into the joint. The purpose of the injection is to 1) improve the lubrication or fluidity of the articulating surfaces of the joint so as to decrease pain or allow more freedom of motion of the joint; and 2) over time, some of the DBM will settle in the damaged cartilage and begin to function as an active scaffold.

### Example 2

The composition of Example 1 can be fortified with the addition of additional materials such as chondroitin and glucosamine as well as additional growth factors, polysaccharides, glycosaminoglycans, growth factor peptides, anti-inflammatory agents, chondrocytes, Human mesenchymal stem cells or other cells capable of differentiating to chrondrocytes and combinations thereof.

### Example 3

Any of the compositions of examples 1-2 can be prepared using about 1 wt. % to about 20 wt. % of a DBM component.

### Example 4a

Non-lubricant, gelling carriers include carbopol polymers and certain alginates, and any of the compositions of examples 1-3 can be prepared using such a gelling agent as a substitute for a lubricant.

### Example 4b

With reference to the compositions of example 4a, such carrier components and others can be used in concentrations of about 0.1% to about 5% by weight.

### Example 5

The addition of the gelling agents is especially useful in that these gels are thixotropic and have high yield strengths, which can assist to maintain the DBM particles in suspension (no mixing required prior to use). Compositions shear thin and, as a result, are very fluid while also being very lubricious. The gel carrier is also useful to allow time release of the DBM or any other active pharmaceutical ingredient (anti-inflammatory agents or pain relief agents).

### Example 6

A gelling carrier is especially advantageous for spinal disk applications, or any place where the possibility of migration needs to be minimized.

### Example 7

A DBM component may be ground to particles of about 25 to about 75 microns in dimension, so that the injection needle size may be between 23 gauge or 14 gauge which will minimize the pain from injection as well as minimize extravasation (leakage). Compositions of any of the preceding examples can be prepared using such DBM particles.

### Example 8

A formulation contains DBM of a particle size between 25 to 50 microns at 1 to 2% by weight, Type 2 collagen particles at a particle size between 25 to 50 microns at 1% by weight, Hyaluronic acid (0.5% solution) and 3 wt % chondroitin sulfate.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. An injectable pharmaceutical composition comprising:
demineralized bone matrix (DBM) particles, at a particle size in a range of about 25 microns to about 75 microns; and
a pharmaceutical carrier.

2. The injectable pharmaceutical composition according to claim 1, wherein the range is from about 25 microns to about 50 microns.

3. The injectable pharmaceutical composition according to claim 1, wherein the particle size is not greater than about 50 microns.

4. The injectable pharmaceutical composition according to claim 1, wherein the DBM is present in a suspension.

5. The injectable pharmaceutical composition according to claim 1, wherein the DBM comprises about 1 wt % to about 99 wt %.

6. The injectable pharmaceutical composition according to claim 5, wherein the DBM comprises about 1 wt % to about 20 wt %.

7. The injectable pharmaceutical composition according to claim 1, wherein the DBM is selected from intact DBM, digested DBM, partially digested DBM, and combinations thereof.

8. The injectable pharmaceutical composition according to claim 7, wherein the digested DBM or the partially digested DBM is obtained from digestion of DBM with a collagenase enzyme.

9. The injectable pharmaceutical composition according to claim 1, wherein the pharmaceutical composition comprises a thixotropic composition.

10. The injectable pharmaceutical composition according to claim 1, wherein the composition is applied to soft tissue.

11. A pharmaceutical composition, comprising:
demineralized bone matrix (DBM) particles at a particle size between about 25 to about 50 microns,
type 2 collagen,
Hyaluroniuc acid, and
an aqueous medium.

12. An injectable pharmaceutical composition suitable for use in soft tissue, said pharmaceutical composition comprising:
demineralized bone matrix (DBM) particles, at a particle size in a range of about 25 microns to about 75 microns; and
a pharmaceutical carrier.

13. The injectable pharmaceutical composition of claim 12 wherein the DBM is present in a suspension.

14. The injectable pharmaceutical composition of claim 12, wherein the pharmaceutical composition comprises at least one adjuvant.

15. The injectable pharmaceutical composition of claim 14, wherein the adjuvant is collagen.

16. The injectable pharmaceutical composition according to claim 12, wherein the DBM is selected from intact DBM, digested DBM, partially digested DBM, and combinations thereof.

17. The injectable pharmaceutical composition of claim 12, further comprising at least one active ingredient.

18. The injectable pharmaceutical composition according to claim 12, wherein the pharmaceutical composition comprises a thixotropic composition.

19. The injectable pharmaceutical composition of claim 12, wherein the particle size is not greater than about 50 microns.

20. The injectable pharmaceutical composition of claim 15, wherein the collagen is human derived collagen.
